(19) United States
(12) Patent Application Publication
(10) Pub. No.: US 2026/0183481 A1
(43) Pub. Date: Jul. 2, 2026

(54) Rotary and Linear Actions Syringe

(71) Applicant: Jamil Mogul, Stockton, CA (US)

(72) Inventor: Jamil Mogul, Stockton, CA (US)

(21) Appl. No.: 19/004,319

(22) Filed: Dec. 28, 2024

(51) Int. Cl. A61M 5/145; A61M 5/315

(52) U.S. Cl. CPC A61M 5/1456; A61M 5/31513

(57) ABSTRACT

A rotary and linear actions syringe, to controllably inject a micro or macro amount of medicinal liquid or any other fluid into a patient, device, equipment, has a barrel with a radial hole that contains a round-headed strip and a spring, which are retained with a pin inserted into the radial hole or that contains a knob with a tip, a plunger that has a helical groove, a circular groove and a linear groove, which are tangentially joined while having a dip between the helical and circular grooves and a ramp between the circular and linear grooves, while the strip freely turns and engages with the grooves, or that only has a helical groove with or from which the knob engages or disengages, and a piston that is attached to the plunger, which is able to be rotated and translated or to be pushed or pulled.

### CROSS REFERENCE TO RELATED APPLICATIONS

Applicant claims priority of provisional application No. 63/617,378, filed Jan. 3, 2024, for “Syringe with Rotary and Linear Actions”.

### BACKGROUND

### Field of the Invention

The present invention relates to an apparatus that provides both rotary and linear actions, one at a time, of the plunger of the syringe without a need for a manual or motorized rotary device in order to inject a micro or macro amount of medicinal liquid, saline or any other liquid into a patient.

### Prior Art

A prevalent practice to achieve micro injection of a medicinal liquid, saline or any other liquid into a patient is to have a controlled micromovement of the plunger of a syringe, which is filled with the liquid, by means of a manual or motorized screw-type mechanism of a rotary device that is attached to the syringe while the device's mechanism is coupled to the syringe's plunger.

Another practice to achieve micro injection of a medicinal liquid, saline or any other liquid into a patient is a syringe pump, which is intended for use on adults, pediatrics, and neonates for transport and which provides for IV and enteral infusions. The syringe pump has an automatic syringe-drive arm and piston brake in addition to having bolus reduction software and wireless integration.

### The Problems Addressed

Manual or motorized rotary devices that are attached to a regular syringe, or software controlled electronic syringe pumps are often intrusive or sometimes ineffective or complicated and their use adds a cost to the medical procedure. This additional cost is passed on to the patients who may not be able to afford it.

### A Solution to the Problems

To address the issues mentioned above, a novel syringe with an option of rotary or linear action, one at a time, of the plunger of the syringe without a need for a manual or motorized rotary device in order to inject a micro or macro amount of medicinal liquid, saline or any other liquid into a patient. This dual action syringe, which would cost almost the same as the regular syringe in high volumes, will be operated like the regular syringe is handled and used. But the novel syringe will now have an inherent. intuitive rotary action in addition to the normal linear (push or pull) action of the plunger.

### BRIEF SUMMARY OF THE INVENTION

In accordance with the present invention, there are two embodiments of the invention, which are briefly stated below.

In the first embodiment, at its proximal end, the barrel of a modified regular syringe has a hole that is partially threaded, but the distal end of the barrel, with the needle, is kept the same. In the modified syringe, the piston is a separate part, which has a stepped proximal end that circumferentially has a groove, while the plunger, which is tubular and has two cross holes at its distal end, is attached to the piston by aligning the piston's groove with the plunger's cross holes and then by press-fitting a pin into each hole. This makes the plunger not only integral to the piston, but also rotatable without causing the piston to rotate, keeping the linear movement/function of the piston intact.

In the modified syringe, the plunger externally has a helical groove whose proximal end tangentially joins a half-circular groove, which, on its other end, joins a longitudinally linear groove. Both the helical and half-circular grooves have the same width and depth. The linear groove has also the same width, but it is deeper, due to which there is a ramp from the end of the half-circular groove to the beginning of linear groove. At the other end of the half-circular groove, there is a curved dip at the joining point of the helical groove. A round-headed strip is inserted, along with a compression spring, into the barrel's threaded hole that takes a set screw. The helical groove provides rotary action while the linear groove allows the linear action.

The spring-loaded round-headed strip engages with the grooves and freely turns due to helical, half-circular and linear grooves and moves up or down, due to the different depths, the ramp, and the curved dip. While turning the plunger, the strip going into the dip gives tactile feedback to the user about which groove is engaged.

In the second embodiment, at its proximal end, the barrel of a modified regular syringe has a threaded hole, but the distal end of the barrel, with the needle, is kept the same, while the tubular plunger of the syringe externally has just a helical groove along with the features for the attachable piston described in the first embodiment. The barrel at its threaded hole has a knob, which has a threaded stem with a round tip that engages with the plunger's helical groove. The user can turn down the knob to engage with the helical groove to achieve the rotary action of the plunger or turn up the knob to disengage from the groove. The disengaged state of the knob allows the linear action of the plunger.

An important advantage of the present invention is that it does not use a manual or motorized rotary device, which is often cumbersome to carry and intrusive to the medical procedure and occupies the space or interferes with or adds steps to the medical procedure.

Another important advantage of the present invention is that it looks and will feel like a regular syringe. As such, it will be operated like the regular syringe is handled and used, but it will now have an inherent, intuitive rotary action in addition to the normal linear (push or pull) action of the plunger.

Yet another important advantage of the present invention is that it would cost almost the same as the regular syringe in high volumes, which would make it affordable for the hospitals and would not add extra cost to the medical procedure for a patient.

These and other advantages of the present invention will become apparent to those skilled in the art after reading the following detailed disclosure of embodiments of the present invention.

### BRIEF DESCRIPTION OF THE VIEWS IN THE DRAWING

### The First Embodiment

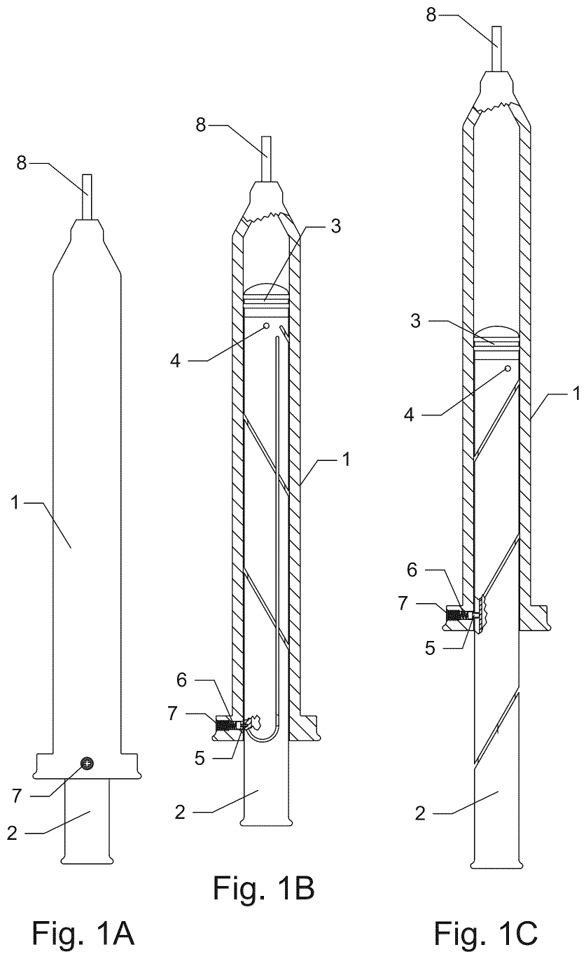

FIG. 1A and FIG. 1B show the front and sectional views, respectively, of the modified syringe, and FIG. 1C shows a sectional view of the modified syringe with the rotated plunger.

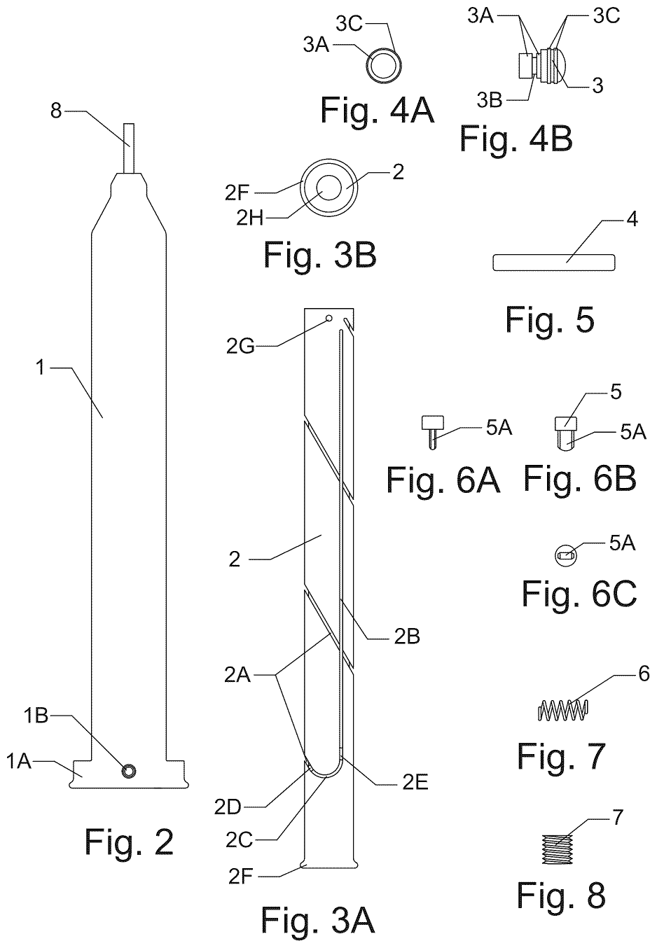

FIG. 2 shows the front view of the barrel of the modified syringe.

FIG. 3A and FIG. 3B show the front and side views, respectively of the tubular plunger of the modified syringe.

FIG. 4A and FIG. 4B show the side and front views of the piston of the modified syringe.

FIG. 5 shows the front view of the pin of the modified syringe.

FIG. 6A, FIG. 6B, and FIG. 6C show the side, front and bottom views of the round-headed strip of the modified syringe, respectively.

FIG. 7 shows the front view of the compression spring of the modified syringe.

FIG. 8 shows the front view of the set screw of the modified syringe.

### The Second Embodiment

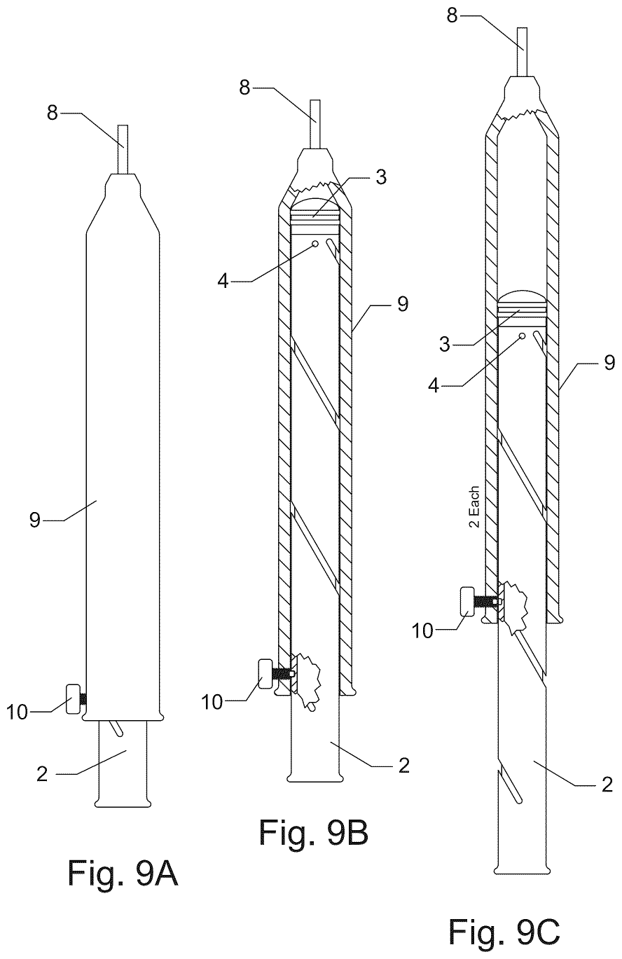

FIG. 9A, FIG. 9B, and FIG. 9C show the front view of the modified syringe and sectional views of the modified syringe and the modified syringe with the rotated plunger.

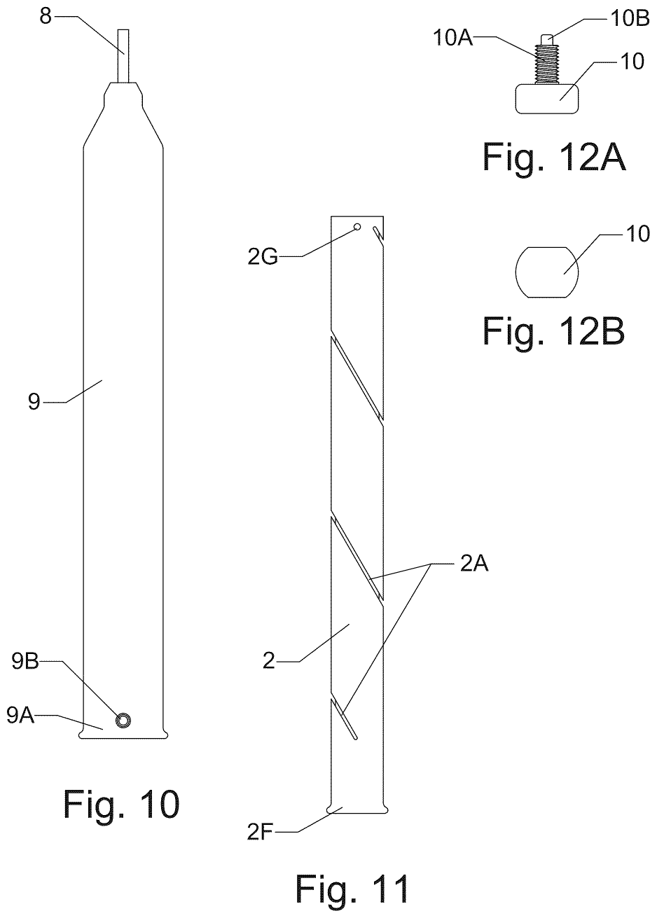

FIG. 10 shows the front view of the barrel of the modified syringe.

FIG. 11 shows the front view of the tubular plunger of the modified syringe.

FIG. 12A and FIG. 12B show the front and bottom views of the knob of the modified syringe, respectively.

### DETAILED DESCRIPTION OF THE EMBODIMENTS

### The First Embodiment

Now, referring to FIG. 1A, FIG. 1B, FIG. 1C, FIG. 2, FIG. 3A, FIG. 3B, FIG. 4A, FIG. 4B, FIG. 5, FIG. 6A, FIG. 6B, FIG. 6C, FIG. 7, and FIG. 8 of the rotary and linear actions syringe in accordance with the present invention, the barrel 1 of a regular syringe with a needle 8, a tubular plunger 2 and a piston 3, is modified to have, on the proximal end 1A of the barrel 1, a partially threaded hole 1B. The tubular plunger 2 and the piston 3 are separated and also modified. The piston 3 has a stepped proximal end 3A that has a circumferential groove 3B, and contains circumferential ribs 3C for an internal seal between the piston 3 and the barrel 1. The tubular plunger 2, which has a grip feature 2F on one end and two cross holes 2G on the other end, is attached to the piston 3 by aligning the circumferential groove 3B with the cross holes 2G and then by press-fitting a pin 4 into each of the cross holes 2G. This makes the tubular plunger 2 not only integral to the piston 3, but also rotatable without causing the piston 3 to rotate, while keeping the linear movement/function of the piston 3 intact.

Additionally, the tubular plunger 2 externally has a helical groove 2A whose proximal end tangentially joins a half-circular groove 2C, which, on its other end, longitudinally joins a linear groove 2B. Both the helical groove 2A and the half-circular groove 2C have the same width and depth. However, the linear groove 2B has also the same width, but it is deeper, due to which there is a ramp 2E going from one end of the half-circular groove 2C to the beginning of the linear groove 2B. At the other end of the half-circular groove 2C, there is a curved dip 2D at the joining point of the helical groove 2A, which provides the rotary action while the linear groove 2B allows the linear action.

A round-headed strip 5 with a flat end 5A is inserted, along with a compression spring 6, into the partially threaded hole 1B of the barrel 1 and retained with a set screw 7 that goes into the partially threaded hole 1B of the barrel 1. The flat end 5A of the spring-loaded round-headed strip 5 engages with and freely turns due to the helical groove 2A, the half-circular groove 2C and the linear groove 2B and moves up or down, due to the different depths, the ramp 3E, and the curved dip 2D. While turning the tubular plunger 2 via the grip feature 2F and the flat end 5A of the round-headed strip 5 going into the dip 2D gives tactile feedback to the user about which groove is engaged.

### The Second Embodiment

Referring to FIG. 9A, FIG. 9B, FIG. 9C, FIG. 10, FIG. 11, FIG. 3B, FIG. 4A, FIG. 4B, FIG. 5, FIG. 12A, and FIG. 12B of the rotary and linear actions syringe in accordance with the present invention, the barrel 9 of a regular syringe with a needle 8, a tubular plunger 2 and a piston 3, is modified to have, on the proximal end 9A of the barrel 1, a threaded hole 9B. Like the first embodiment, the tubular plunger 2 and the piston 3 are separated and also modified. As indicated above, the piston 3 has a stepped proximal end 3A that has a circumferential groove 3B, and also has circumferential ribs 3C for an internal seal between the piston 3 and the barrel 9. The tubular plunger 2, which has a grip feature 2F on one end and two cross holes 2G on the other end, is attached to the piston 3 by aligning the circumferential groove 3B with the cross holes 2G and then by press-fitting a pin 4 into each of the cross holes 2G. This makes the tubular plunger 2 not only integral to the piston 3, but also rotatable without causing the piston 3 to rotate, while keeping the linear movement/function of the piston 3 intact. Additionally, the tubular plunger 2 externally has a helical groove 2A which provides the rotary action.

A knob 10, which has a threaded stem 10A and a rounded tip 10B, is screwed into the threaded hole 9B of the barrel 9. The rounded tip 10B of the knob 10 engages with the helical groove 2A of the tubular plunger 2. The user can turn down the knob 10 to engage with the helical groove 2A to achieve the rotary action of the tubular plunger 2 or turn up the knob 10 to disengage from the helical groove 2A. The disengaged state of the knob 10 allows the linear action of the tubular plunger 2.

The invention claimed is:

1. A rotary and linear actions syringe, to controllably inject a micro or macro amount of medicinal liquid or saline or any other fluid into a human or animal patient, device, apparatus, or equipment, said syringe comprising:
a barrel having at its one end a radial hole that is plain or threaded, said radial hole having a strip and a spring, which are retained with a plain or threaded pin that is inserted into said radial hole, and said strip having a shaped head at one end;
a plunger having at one end a receptacle hole and a cross hole, said plunger having a helical groove, a circular groove, and a linear groove, said helical groove joining said circular groove at a point of said circular groove, said point having a geometrically shaped dip that joins said helical groove and said circular groove, said linear groove joining said circular groove at a second point of said circular groove, said second point having a geometrically shaped ramp that joins said circular groove and said linear groove, and said strip freely turning and engaging with said helical groove, said dip, said circular groove, said ramp, and said linear groove;
a piston having a circumferential groove in addition to having normal sealing ribs, said piston being attached to said plunger by inserting said piston into said receptacle hole of said plunger and retaining said piston via a pin that is inserted into said cross hole of said plunger, said pin engaging with said circumferential groove while allowing said plunger to freely rotate and translate or to be pushed or pulled, and said ribs providing an internal seal between said piston and said barrel.

2. Said barrel having in said radial hole a knob with a plain or threaded stem that has a tip, said plunger having said helical groove only, and said tip engaging with or disengaging from said helical grove by turning said knob down or up while allowing said plunger to freely rotate and translate or to be pushed or pulled.
